(19) United States
(12) Patent Application Publication
(10) Pub. No.: US 2017/0370950 A1
(43) Pub. Date: Dec. 28, 2017

(54) BIOTIN CONJUGATES OF ANALYTES CONTAINING AMINO, HYDROXYL, OR THIOL FUNCTIONAL GROUPS FOR USE IN IMMUNODIAGNOSTIC ASSAYS

(71) Applicant: Calbiotech, Inc., El Cajon, CA (US)

(72) Inventor: Yonas YOHANNES, San Diego, CA (US)

(21) Appl. No.: 15/533,607

(22) PCT Filed: Dec. 8, 2015
(86) PCT No.: PCT/IB2015/002471

§ 371 Date: Jun. 6, 2017

(51) Int. Cl. G01N 33/74; G01N 33/78; C07D 495/04; G01N 33/543; G01N 33/92; G01N 33/82

(52) U.S. Cl. CPC G01N 33/743; G01N 33/92; G01N 33/82; C07D 495/04; G01N 33/54393; G01N 33/78

(57) ABSTRACT

The present disclosure provides a compound having Formula (I): or a pharmaceutically acceptable salt of hydrate thereof, wherein R¹, A, m, and n are as described herein, for use in conducting immunodiagnostic assays for accurately measuring concentrations of circulating vitamin D, as well as for thyroxine and related analytes, estrogen and related analytes, and testosterone and related analytes.

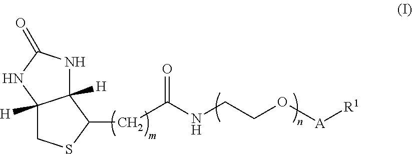

### CROSS-REFERENCE TO RELATED APPLICATION

This application claims the benefit of priority to U.S. patent application Ser. No. 14/563,691, filed Dec. 8, 2014 and titled “BIOTIN CONJUGATES OF ANALYTES CONTAINING AMINO, HYDROXYL, OR THIOL FUNCTIONAL GROUPS FOR USE IN IMMUNODIAGNOSTIC ASSAYS,” the content of which is hereby incorporated by reference in its entirety.

### TECHNICAL FIELD OF THE INVENTION

The present disclosure relates to biotin conjugates, and in particular, to compounds, compositions, and methods of preparing and using biotin conjugates of analytes containing amino, hydroxyl, or thiol functional groups for use in immunodiagnostic assays.

### BACKGROUND OF THE INVENTION

Conjugates of biotin are often utilized in enzyme-linked immunosorbent assays (ELISAs). Of particular interest are ELISAs for analytes in the body such as vitamin D and related analytes, thyroxine and related analytes, estrogen and related analytes, and testosterone and related analytes. Vitamin D refers to a group of fat-soluble secosteroids which enhance intestinal absorption of calcium, iron, magnesium, phosphate, and zinc. In humans, the most important compounds in this group are vitamin D2 (ergocalciferol) and vitamin D3 (cholecalciferol). Vitamin D2 exists in nature and is primarily obtained from diet and supplementation and is metabolized in the body to 25-hydroxyvitamin D2. Vitamin D3 is also a seco-steroid that is synthesized in the skin and undergoes sequential metabolic conversion to 25-hydroxyvitamin D3 in the liver and to 1-,25-dihydroxyvitamin D3 in the kidney. The “total 25-hydroxyvitamin D” refers to the sum of the concentrations of 25-hydroxyvitamin D2 and 25-hydroxyvitamin D3, in serum or plasma.

Accurate monitoring of total 25-hydroxyvitamin D level is critical in clinical settings. The level of the total 25-hydroxyvitamin D in the blood serves as a good indicator of vitamin D deficiency and can be used to guide therapies for avoiding medical conditions arising from hypovitaminosis D. The biosynthesis and metabolism of 25-hydroxyvitamin D and 1-, 25-dihydroxyvitamin D are regulated by the factors that control mineral and skeletal metabolism. Therefore, the levels of 25-hydroxyvitamin D and 1-, 25-dihydroxyvitamin D in the serum is an important pathophysiological indicator in several diseases, for example, vitamin D dependent rickets types I and II. Insufficient endogenous production coupled with insufficient dietary supplementation and the inability of the small intestine to absorb required amounts of vitamin D from food usually results in hypophosphatemia, hypocalcemia and possibly secondary hyperparathyroidism.

Given its pathophysiological importance tremendous efforts have been directed towards developing assays for accurately measuring concentrations of circulating vitamin D and related analytes, as well as for other circulating hormones including, for example, thyroxine and related analytes, estrogen and related analytes, and testosterone and related analytes. As such, there is a need to develop simpler, effective conjugates to successfully conduct ELISAs for such analytes in a time and cost efficient manner.

### SUMMARY OF THE INVENTION

The present disclosure addresses these needs and more by providing compounds, compositions and methods for conducting immunodiagnostic assays for accurately measuring concentrations of circulating vitamin D, as well as for thyroxine and related analytes, estrogen and related analytes, and testosterone and related analytes.

According to the present disclosure, some technical effects may be achieved in part by a compound having Formula I:

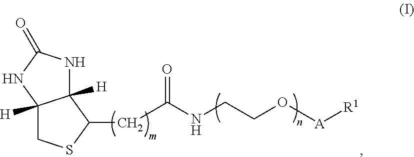

or a pharmaceutically acceptable salt or hydrate thereof, wherein:

A is —(CH₂)_{q}—, —(CH₂)_{q}O(CH₂)ₜ—, —(CH₂)_{q}NR²(CH₂)_{q}—, —(CH₂)_{q}S(O)ᵤ(CH₂)_{q}—, —(CH₂)ₜC(O)NR²(CH₂)ₜ—, —(CH₂)_{q}NR²C(O)(CH₂)ₜ—, —(CH₂)_{q}NR²C(O)O(CH₂)ₜ—, —(CH₂)_{q}NR²C(O)NR³(CH₂)ₜ—, or —(CH₂)_{q}NR²C(O)S(CH₂)_{q}—;

R¹ is vitamin D, thyroxine, estrogen or testosterone or a metabolite or derivative thereof;

R² and R³ are each independently hydrogen or (C₁-C₆)alkyl;

m is an integer number selected from 4, 5 and 6; n is an integer number selected from 2 to 36; q is an integer number selected from 1 to 12; t is an integer number selected from 0 to 12; and u is an integer number selected from 0, 1 and 2.

According to the present disclosure, some technical effects may be achieved in part by a solid phase assay method for detecting vitamin D, thyroxine, estrogen or testosterone or a metabolite or derivative thereof in a sample, by:

a) providing a solid phase support having immobilized thereon a protein or antibody that binds to the vitamin D, thyroxine, estrogen or testosterone or a metabolite or derivative thereof in the sample, and a compound having Formula I or a pharmaceutically acceptable salt or hydrate thereof;

b) contacting the solid phase support with the sample of the vitamin D, thyroxine, estrogen or testosterone or a metabolite or derivative thereof, and the compound of Formula I, for a time sufficient to allow binding to the protein or antibody attached to the solid phase support;

c) contacting the solution and solid phase support obtained in step (b) with streptavidin-horse radish peroxidase (SA-HRP), for a time sufficient to effect conjugation of the SA-HRP to the compound of Formula I, which may be unbound or bound to the protein or antibody attached to the solid phase support;

d) removing the solution obtained in step (c) containing unbound SA-HRP conjugated to the compound of Formula I; and

e) detecting the presence of bound SA-HRP conjugated to the compound of Formula

wherein the amount of the bound SA-HRP conjugated to the compound of Formula I in step e) is inversely proportional to the amount of the vitamin D, thyroxine, estrogen or testosterone or a metabolite or derivative thereof in the test sample.

According to the present disclosure, some technical effects may be achieved in part by a kit for determining the concentration of vitamin D, thyroxine, estrogen or testosterone or a metabolite or derivative thereof in a sample of human serum by an immune-based competitive protein binding assay, which includes:

a) a standardized quantity of a compound of Formula I or a standardized solution of a compound of Formula I, or a pharmaceutically acceptable salt or hydrate thereof; and

b) a standardized quantity of a protein or antibody that specifically binds to vitamin D, thyroxine, estrogen or testosterone or a metabolite or derivative thereof, and the compound having Formula I, or a pharmaceutically acceptable salt or hydrate thereof.

Additional aspects and other features of the present disclosure will be set forth in the description which follows and in part will be apparent to those having ordinary skill in the art, upon examination of the following or may be learned from the practice of the present disclosure. Additional aspects and technical effects of the present disclosure will become readily apparent to those skilled in the art from the following detailed description wherein embodiments of the present disclosure are described simply by way of illustration of the best mode contemplated to carry out the present disclosure. As will be realized, the present disclosure is capable of other and different embodiments, and its several details are capable of modifications in various obvious respects, all without departing from the present disclosure. Accordingly, the drawings and description are to be regarded as illustrative in nature, and not as restrictive. The advantages of the present disclosure may be realized and obtained as particularly pointed out in the appended claims.

### DETAILED DESCRIPTION OF THE INVENTION

### Definitions

Abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—, —C(O)O— is equivalent to —OC(O)—, and —C(O)NR— is equivalent to —NRC(O)—, and the like.

The term “alkyl,” by itself or as part of another substituent, means, unless otherwise stated, a straight (i.e., unbranched) or branched chain, or cyclic hydrocarbon radical, or combination thereof, which may be fully saturated, mono- or polyunsaturated and can include di- and multivalent radicals, having the number of carbon atoms designated, e.g., C₁-C₁₀ means 1 to 10 carbons; and C₁-C₆ means 1 to 6 carbons.

Examples of saturated hydrocarbon radicals include, but are not limited to, groups such as methyl, ethyl, n-propyl, isopropyl, n-butyl, sec-butyl, isobutyl, t-butyl, n-pentyl, n-hexyl, n-heptyl, n-octyl, cyclohexyl, (cyclohexyl)methyl, cyclopropylmethyl, and the like.

An unsaturated alkyl group is one having one or more double bonds or triple bonds.

Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule.

Examples of cycloalkyl include, but are not limited to, cyclopentyl, cyclohexyl, 1-cyclohexenyl, cycloheptyl, and the like.

Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “alkylene” by itself or as part of another substituent means a divalent radical derived from an alkyl, as exemplified but not limited by —CH₂CH₂CH₂CH₂—. Typically, an alkyl or alkylene group will have from 1 to 24 carbon atoms, with those groups having 10 or fewer carbon atoms being common. A “lower alkyl” or “lower alkylene” is a shorter chain alkyl or alkylene group, generally having 1 to 6 carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein nitrogen and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule.

Included within the definition of heteroalkyl compounds are alkoxy, thioalkoxy, aminoalkyl, aminodialkyl and the like. Examples include, but are not limited to, —OCH₃, —CH₂OCH₃, —CH═CHOCH₃, —SCH₃, —CH₂SCH₃, —S(O)₂CH₃, —NHCH₃, —N(CH₃)₂ —CH₂N(CH₃)₂, —CH═CHN(CH₃)₂.

Similarly, the term “heteroalkylene” by itself or as part of another substituent means a divalent radical derived from heteroalkyl, as exemplified, but not limited by, —CH₂CH₂SCH₂CH₂— and —CH₂SCH₂CH₂NHCH₂—. For heteroalkylene groups, heteroatoms can also occupy either or both of the chain termini, e.g., alkyleneoxo, alkylenedioxo, alkyleneamino, alkylenediamino, and the like.

As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (from 1 to 3 rings) which are fused together or linked covalently.

The term “heteroaryl” refers to aryl groups (or rings) that contain from 1 to 4 heteroatoms selected from N, O, and S, wherein nitrogen and sulfur atoms are optionally oxidized, and the nitrogen atom(s) are optionally quaternized. A heteroaryl group can be attached to the remainder of the molecule through a carbon or heteroatom.

Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridinyl, 3-pyridinyl, 4-pyridinyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Substituents for each of above noted aryl and heteroaryl ring systems are selected from the group of acceptable substituents described below.

The terms “arylene” and “heteroarylene” refer to the divalent derivatives of aryl and heteroaryl, respectively.

The term “arylalkyl” is meant to include those radicals in which an aryl group is attached to an alkyl group, e.g., benzyl, phenethyl, pyridinylmethyl and the like, including those alkyl groups in which a carbon atom, e.g., a methylene group, has been replaced by, for example, an oxygen atom, e.g., phenoxymethyl, 2-pyridinyl-oxymethyl, 3-(1-naphthyloxy)propyl, and the like.

Similarly, the term “heteroarylalkyl” is meant to include those radicals in which a heteroaryl group is attached to an alkyl group, e.g., pyridinylmethyl, quinolinylmethyl, 1,2,4-triazolyl[4,3-b]pyridazinyl-methyl, 1H-benzotriazolylmethyl, benzothiazolylmethyl, and the like. However, the term “haloaryl,” as used herein is meant to cover only aryls substituted with one or more halogens.

The term “oxo” as used herein means an oxygen that is double bonded to a carbon atom.

Each of above terms, e.g., “alkyl,” “heteroalkyl,” “cycloalkyl,” “heterocycloalkyl”, “aryl,” and “heteroaryl,” as well as their divalent radical derivatives, are meant to include both substituted and unsubstituted forms of the indicated radical. Possible substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, —NR′R″, —SR′, -halogen, —SiR′R″R″′, —OC(O)R′, —C(O)R′, —CO₂R′, —C (O)NR′R″, —OC (O)NR′, —NR′C(O)R″, —NR′C(O)NR″R″′, —NR′C(O)OR″, —NRC(NR′R″)═NR″′, —S(O)R′, —S(O)₂R′, —S(O)₂NR′R″, —NRSO₂R′, —CN and —NO₂.

R′, R″, R′″ and R″″ each independently refer to hydrogen, substituted or unsubstituted heteroalkyl, substituted or unsubstituted cycloalkyl, substituted or unsubstituted heterocycloalkyl, substituted or unsubstituted aryl, e.g., aryl substituted with 1-3 halogens, substituted or unsubstituted alkyl, alkoxy or thioalkoxy groups, or arylalkyl groups. When a compound of the disclosure includes more than one R group, for example, each of the R groups is independently selected as are each R′, R″, R″′ and R″″ groups when more than one of these groups is present.

When R′ and R″ are attached to the same nitrogen atom, they can be combined with the nitrogen atom to form 4-, 5-, 6-, or 7-membered ring. For example, —NR′R″ is meant to include, but not be limited to, 1-pyrrolidinyl and 4-morpholinyl.

From above discussion of substituents, one of skill in art will understand that the term “alkyl” is meant to include groups including carbon atoms bound to groups other than hydrogen groups, such as haloalkyl, e.g., —CF₃ and CH₂CF₃, and acyl, e.g., —C(O)CH₃, —C(O)CF₃, —C(O)CH₂OCH₃, and the like.

Similar to the substituents described for alkyl radicals above, exemplary substituents for aryl and heteroaryl groups as well as their divalent derivatives are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR′C(O)R″, —NR′C(O)NR″R″′, —NR′C(O)OR″, —NRC(NR′R″R″′)═NR″″, —NRC(NR′R″)═NR″′, —S(O)R′, —S(O)₂R′, —S(O)₂NR′R″, —NRSO₂R′, —CN, NO₂, —N₃, —CH(Ph)₂, and fluoro(C₁-C₄)alkyl, in a number ranging from zero to the total number of open valences on the aromatic ring system. R′, R″, R″′ and R″″ are each independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

When a compound of the disclosure includes more than one R group, for example, each of the R groups is independently selected as are each R′, R″, R″′ and R″″ groups when more than one of these groups is present.

As used herein, the term “heteroatom” or “ring heteroatom” is meant to include oxygen (O), nitrogen (N), sulfur (S), phosphorus (P), and silicon (Si).

The compounds of the present disclosure may exist as salts. The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent.

Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like {see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)- , or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine¹²⁵ or carbon-14 (¹⁴C). All isotopic variations of the compounds of the present disclosure, whether radioactive or not, are encompassed within the scope of the present disclosure.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

The description of the compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, physiological conditions.

The symbol “-” or “˜”denotes the point of attachment of a moiety to the remainder of the molecule.

In the following description, for the purposes of explanation, numerous specific details are set forth in order to provide a thorough understanding of exemplary embodiments. It should be apparent, however, that exemplary embodiments may be practiced without these specific details or with an equivalent arrangement. In other instances, well-known structures and devices are shown in order to avoid unnecessarily obscuring the exemplary embodiments. In addition, unless otherwise indicated, all numbers expressing quantities, ratios, and numerical properties of ingredients, reaction conditions, and so forth used in the specification and claims are to be understood as being modified in all instances by the term “about.”

### Compounds, Compositions and Methods

The present disclosure is directed to compounds, compositions and methods for conducting immunodiagnostic assays for accurately measuring concentrations of circulating hormones in the human body. Circulating hormones include those having amino, hydroxyl, and thiol functionalities, for example, vitamin D and related analytes, thyroxine and related analytes, estrogen and related analytes, and testosterone and related analytes. The disclosed compounds are conjugated to biotin or a derivative thereof, which can be used in an immunodiagnostic assay, for example, an enzyme-linked immunoassay (ELISA), for determining the presence and quantity of the analyte in question. In addition to ELISAs, the disclosure includes and could be employed as a quantitative method for analyte measurement using any of the following assays: enzyme immunoassay (EIA), membrane immunobead assay (MIA), radioimmunoassay (RIA), immunoradiometric assay (IRMA), line immunoassay (LIA) or immunoluminometric assay (ILMA), fluorescent immunosorbent assay (FIA), or immunofluorometric assay (FIA), which are set up for manual or semi-automated or fully automated operations.

Of particular interest are ELISAs for analytes such as vitamin D2, 1-hydroxyvitamin D2, 25-hydroxyvitamin D2, 1-,25-dihydroxyvitamin D2, vitamin D3, 1-hydroxyvitamin D3, 25-hydroxyvitamin D3, and 1-,25-dihydroxyvitamin D3, thyroxine and related analytes, estrogen and related analytes, and testosterone and related analytes, etc. The disclosure provides specific biotin conjugates, a novel process for synthesizing and isolating such biotin conjugates, and the use of these conjugates to detect specific classes of hormone analytes that contain an amino, hydroxyl, or thiol functionality.

Biotin or a derivative thereof, can be conjugated to an analyte compound through a linker, for example, a polyethylene glycol (PEG) linking group. The resulting biotin-conjugated PEG linker can then be directly attached to a specific analyte or a derivative thereof via an amino, hydroxyl or thiol functionality on the analyte. In one non-limiting example, one of the linkers provided herein is a PEG 12 linker. The PEG 12 linker can be attached to biotin on one end through an amide functionality, and can be attached to a specific analyte on the other end through a suitably functionalized chemical handle, for example, a carboxylic acid derivative, isocyanate derivative, thioisocyanate derivative, hydroxyl derivative and the like. The functionalized chemical handle facilitates conjugation of the PEG linker to the amino, hydroxyl or thiol functionality of the analyte using known synthetic procedures. Alternatively, the biotin-conjugated PEG linker can be attached to a specific analyte or a derivative thereof, through one or more organic moieties, for example, an aminopropyl group, which in turn is attached to a specific analyte or a derivative thereof via an amino, hydroxyl or thiol functionality.

PEG is a polyether compound having the following repeating structure: H—(OCH₂—CH₂)—OH, where “n” is an integer number. The numbers that are often included in the names of PEGs indicate their average molecular weights (e.g. a PEG with n=9 would have an average molecular weight of approximately 400 daltons, and would be labeled PEG 400). Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn), which can be measured by mass spectrometry. PEGs can be prepared by polymerization of ethylene oxide and are commercially available over a wide range of molecular weights from 300 g/mol to 10,000,000 g/mol. Depending on their molecular weight, PEGs can be liquids or low-melting solids. PEGs with different molecular weights find use in different applications due to their different physical properties, e.g., viscosity, due to the different length of their chains.

In contrast to other linking groups known in the art, it has been found that the length of the PEG linker between biotin or a derivative thereof, and an analyte of interest, can vary from about 2 nm (PEG, n=2) to 14 nm (PEG, n=36) for the repeating H—(OCH₂—CH₂)ₙ—OH polymeric unit. In other embodiments, n can vary from about n=4 to n=30, or from about n=6 to n=24, or from about n=8 to n=18 or from about n=10 to n=12 (˜5.6 nm) and provide good results during an immunodiagnostic assay.

The use of PEG linkers within this range of length is unexpected since it was generally accepted and understood in the art that if the linking group was too long, there was a higher probability for the linking group to assume conformations in solution that are detrimental to the binding of the targeted antibody as well as the binding of streptavidin. The importance of efficient binding of both the targeted antibody to the analyte of interest as well as streptavidin to biotin is extremely important in order to obtain a reproducible and precise immunodiagnostic assay. In addition, a relatively longer PEG linker provides increased water solubility of the reactant analyte.

While the disclosure describes vitamin D derivatives or derivatives of other analytes (e.g., thyroxine, estrogen, estradiol, testosterone, etc.) in general that have a primary or secondary amino, hydroxyl or thiol group that can be employed in a immunodiagnostic assay such as an ELISA, it is also important that the derivatives not compromise the overall efficacy and sensitivity of the assay. Therefore it is generally understood that the derivatives meet the following criteria:

For the each analyte derivative the sensitivity level of detection exists which is greater than or optionally lies in a lower range of concentration than the concentration of analyte metabolite which is present in the sample that is being tested.

Each analyte derivative is stable in plasma, serum, saliva or urine or any other sample composition that is tested and is not susceptible to degrading enzymes if any present therein.

Each analyte derivative is stable to the light, temperature and required storage conditions for periods that are consider acceptable and meet the perquisite industry standards for shelf life.

These criteria are achieved by analyte derivatives such as those exemplified by the formulas shown in the embodiments disclosed herein.

Thus, in one or more embodiments, the disclosure provides a compound having Formula I:

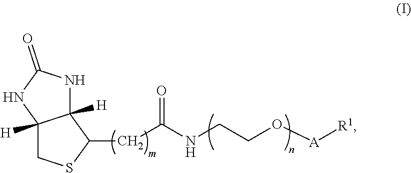

or a pharmaceutically acceptable salt or hydrate thereof, wherein:

A is —(CH₂)_{q}—, —(CH₂)_{q}O(CH₂)ₜ—, —(CH₂)_{q}NR²(CH₂)_{q}—, —(CH₂)_{q}S(O)ᵤ(CH₂)_{q}—, —(CH₂)ₜC(O)NR²(CH₂)ₜ—, —(CH₂)_{q}NR²C(O)(CH₂)ₜ—, —(CH₂)_{q}NR²C(O)O(CH₂)ₜ—, —(CH₂)_{q}NR²C(O)NR³(CH₂)ₜ—, or —(CH₂)_{q}NR²C(O)S(CH₂)_{q}—;

R¹ is vitamin D, thyroxine, estrogen or testosterone or a metabolite or derivative thereof;

R² and R³ are each independently hydrogen or (C₁-C₆)alkyl;

m is an integer number selected from 4, 5 and 6;

n is an integer number selected from 2 to 36;

q is an integer number selected from 1 to 12;

t is an integer number selected from 0 to 12; and

u is an integer number selected from 0, 1 and 2.

In other embodiments, the disclosure provides a compound of Formula I, wherein R₁ has Formula II:

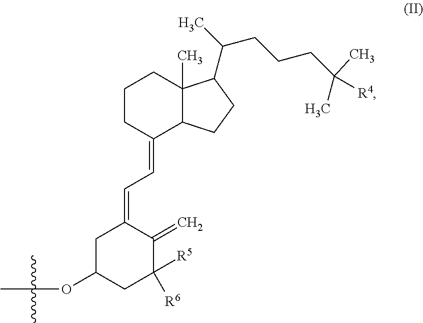

or a pharmaceutically acceptable salt or hydrate thereof, wherein:

R⁴ is hydrogen or hydroxyl; and

R⁵ and R⁶ are each independently hydrogen, hydroxyl, (C₁-C₆)alkyl, or (C₁-C₆)alkoxy;

In other embodiments, the disclosure provides a compound of Formula I, wherein R¹ is 25-hydroxyvitamin D₂ or 25-hydroxyvitamin D₃.

In other embodiments, the disclosure provides a compound of Formula I, wherein R¹ has Formula III:

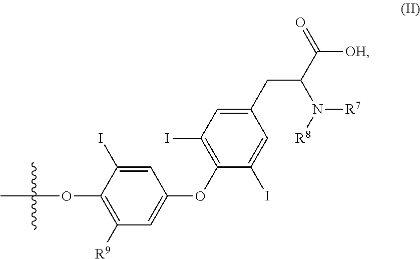

or a pharmaceutically acceptable salt or hydrate thereof, wherein:

R⁷ and R⁸ are each independently hydrogen or (C₁-C₆)alkyl; and

R⁹ is hydrogen or iodine, or

R¹ has Formula IV:

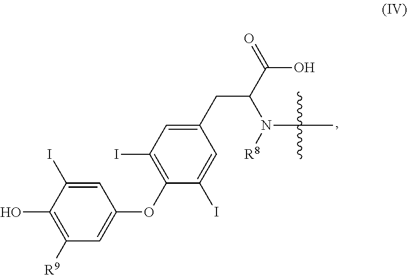

or a pharmaceutically acceptable salt or hydrate thereof, wherein:

R⁷ is hydrogen or (C₁-C₆)alkyl; and

R⁹ is hydrogen or iodine.

In other embodiments, the disclosure provides a compound of Formula I, wherein R¹ is triidothyronine (T₃) or thyroxine (T₄).

In other embodiments, the disclosure provides a compound of Formula I, wherein R¹ has Formula V, VI, VII, VIII, IX, X, or XI:

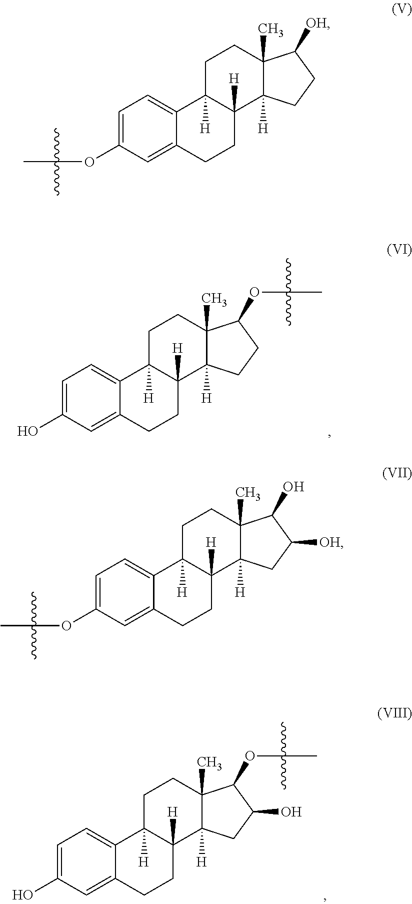

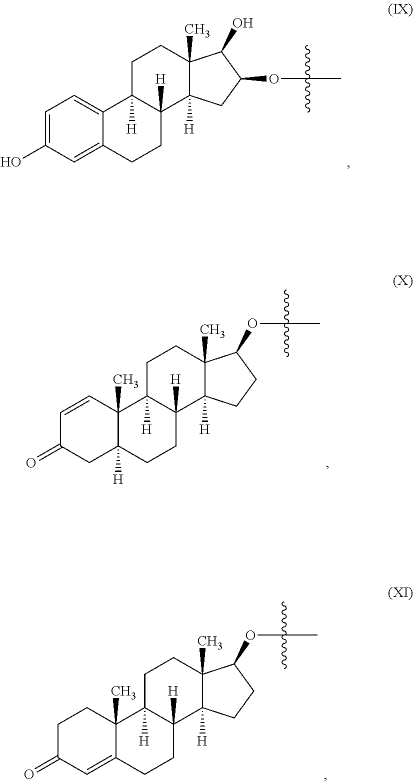

or a pharmaceutically acceptable salt or hydrate thereof.

In other embodiments, the disclosure provides a compound of Formula I, wherein R¹ is estrone (E₁), estradiol (E₂), estriol (E₃), 1-testosterone (1-T), or testosterone.

In other embodiments, the disclosure provides a compound of Formula I, wherein A is —(CH₂)₃—, —(CH₂)₃O(CH₂)₃₋₆—, —(CH₂)₃NR²(CH₂)₃₋₆—, —(CH₂)₃S(CH₂)₃₋₆—, —(CH₂)₂C(O)NR²(CH²)₃₋₆—, —(CH₂)_{q}NR²C(O)(CH₂)₃₋₆—, —(CH₂)_{q}NR²C(O)O(CH₂)₃₋₆—, —(CH₂)_{q}NR²C(O)S(CH₂)₃₋₆—, —(CH₂)_{q}NR²C(O)NR²(CH₂)₃₋₆—, or —(CH₂)₂NR²C(O)—.

In one or more embodiments, the disclosure provides a solid phase assay method for detecting vitamin D, thyroxine, estrogen or testosterone or a metabolite or derivative thereof in a sample, the method including:

a) providing a solid phase support having immobilized thereon a protein or antibody that binds to the vitamin D, thyroxine, estrogen or testosterone or a metabolite or derivative thereof in the sample, and a compound having Formula I, or a pharmaceutically acceptable salt or hydrate thereof;

b) contacting the solid phase support with the sample of the vitamin D, thyroxine, estrogen or testosterone or a metabolite or derivative thereof, and the compound of Formula I, for a time sufficient to allow binding to the protein or antibody attached to the solid phase support;

c) contacting the solution and solid phase support obtained in step (b) with streptavidin-horse radish peroxidase (SA-HRP), for a time sufficient to effect conjugation of the SA-HRP to the compound of Formula I, which may be unbound or bound to the protein or antibody attached to the solid phase support;

d) removing the solution obtained in step (c) containing unbound SA-HRP conjugated to the compound of Formula I; and

e) detecting the presence of bound SA-HRP conjugated to the compound of Formula I,

wherein the amount of the bound SA-HRP conjugated to the compound of Formula I in step e) is inversely proportional to the amount of the vitamin D, thyroxine, estrogen or testosterone or a metabolite or derivative thereof in the test sample.

In other embodiments, the disclosure provides a solid phase assay method for detecting vitamin D, thyroxine, estrogen or testosterone or a metabolite or derivative thereof in a sample, wherein vitamin D includes 25-hydroxyvitamin D2 and 25-hydroxyvitamin D3.

In other embodiments, the disclosure provides a solid phase assay method for detecting vitamin D, thyroxine, estrogen or testosterone or a metabolite or derivative thereof in a sample, wherein detecting the presence of bound SA-HRP conjugated to the compound of Formula I is accomplished through absorbance measured spectrophotometrically at 450 nm.

In other embodiments, the disclosure provides a solid phase assay method for detecting vitamin D, thyroxine, estrogen or testosterone or a metabolite or derivative thereof in a sample, wherein detecting the presence of bound SA-HRP conjugated to the compound of Formula I is accomplished by adding a solution of TMB (3,3′,5,5′-tetramethylbenzidien) reagent to the bound SA-HRP conjugated to the compound of Formula I and incubating at room temperature to develop a blue color; stopping the blue color development by the addition of a stop solution (0.16N sulfuric acid); and measuring absorbance spectrophotometrically at 450 nm against a standard curve.

In other embodiments, the disclosure provides a solid phase assay method, wherein the assay method is an enzyme immunoassay, an enzyme-linked immunosorbent assay, a radioimmunoassay, an immunoradiometric assay, a luminescence assay, a fluorescence immunoassay, or an immunofluorometric assay.

In one or more embodiments, the disclosure provides a kit for determining the concentration of vitamin D, thyroxine, estrogen or testosterone or a metabolite or derivative thereof in a sample of human serum by an immune-based competitive protein binding assay, which includes:

a) a standardized quantity of a compound of Formula I or a standardized solution of a compound of Formula I, or a pharmaceutically acceptable salt or hydrate thereof; and

b) a standardized quantity of a protein or antibody that specifically binds to vitamin D, thyroxine, estrogen or testosterone or a metabolite or derivative thereof, and the compound having Formula I, or a pharmaceutically acceptable salt or hydrate thereof.

In other embodiments, the disclosure provides a kit including a solid phase support having immobilized thereon the standardized quantity of a protein or antibody that specifically binds to vitamin D, thyroxine, estrogen or testosterone or a metabolite or derivative thereof, and the compound having Formula I.

In other embodiments, the disclosure provides a kit including a solid phase support selected from a microtitration plate, a microparticle, a polymeric material, and cellulose.

In other embodiments, the disclosure provides a kit including streptavidin-horse radish peroxidase (SA-HRP).

In other embodiments, the disclosure provides a kit, wherein the competitive protein binding assay is an enzyme immunoassay, an enzyme-linked immunosorbent assay, a radioimmunoassay, an immunoradiometric assay, a luminescence assay, a fluorescence immunoassay or an immunofluorometric assay.

In other embodiments, the disclosure provides methods for synthesizing a compound of Formula I, by:

a) dissolving an n-hydroxysuccinimide-PEG-biotin complex in an alcohol solvent such as methanol or other suitable organic solvent to make a first solution;

dissolving an analyte with an amino, hydroxy or a thiol functional group in N,N-DMF or other suitable polar solvent to make a second solution;

mixing the first solution with the second solution, and reacting the n-hydroxysuccinimide-PEG-biotin complex with the analyte with an amino, hydroxy or a thiol functional group to provide the compound of Formula I; and

isolating the compound of Formula I using either a dialysis or a chromatographic separation method.

The following examples illustrate the preparation and use of the compounds of Formula I.

### EXAMPLES

The synthesis of the compounds of Formula I can be performed as described below in Schemes I to V.

### Example I

### General Synthetic Procedures for the Synthesis of the Compounds of Formula I having Structure A

In one embodiment, the compound of Formula I has structure A:

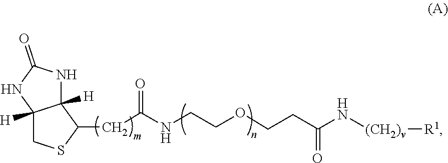

where m is an integer selected from 4, 5, and 6; n is an integer selected from 2 to 36; v is an integer selected from 1 to 6; and R¹ is 3-O-vitamin D (e.g., 3-O-vitamin D2, 3-O-1-hydroxyvitamin D2, 3-O-25-hydroxyvitamin D2, 3-O-1-,25-dihydroxyvitamin D2, 3-O-vitamin D3, 3-O-1-hydroxyvitamin D3, 3-O-25-hydroxyvitamin D3, and/or 3-O-1-,25-dihydroxyvitamin D3), thyroxine and related analytes, estrogen and related analytes, and testosterone and related analytes.

The synthesis of the compound of Formula I having structure A is shown below in Scheme I.

Compound 1 (commercially available from Thermo Scientific, Inc.) is reacted with NH₂(CH₂)₃-(3)-O-vitamin D (commercially available from Toronto Research Chemicals, Inc.) to provide compound 2. Compound 2 can be purified by dialysis or other chromatographic techniques, for example, silica gel gravity column chromatography, reverse or normal phase HPLC or UPLC, or simple extraction/isolation techniques routinely used for isolation/purification for small to medium sized organic molecules. Other compounds having Formula I and structure A can be made by using the appropriate starting materials as known by those of skill in the art.

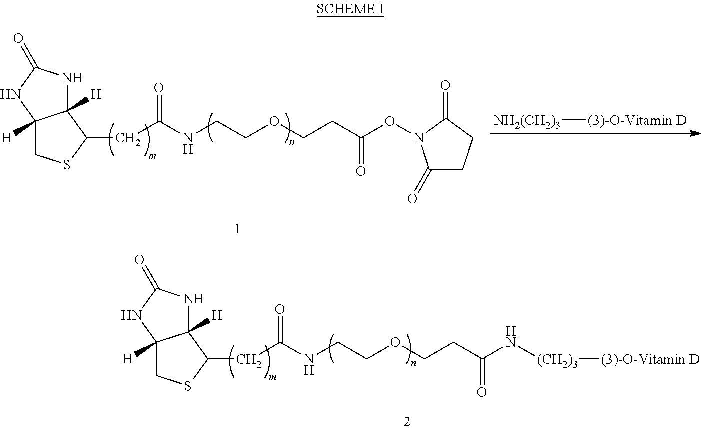

### Example II

### General Synthetic Procedures for the Synthesis of the Compounds of Formula I having Structure B

In another embodiment, the compound of Formula I has structure B:

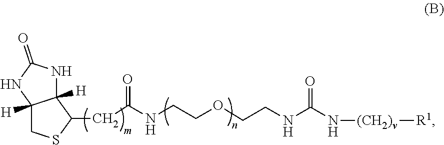

where m is an integer selected from 4, 5, and 6; n is an integer selected from 2 to 36; v is an integer selected from 1 to 6; and R¹ is 3-O-vitamin D (e.g., 3-O-vitamin D2, 3-O-1-hydroxyvitamin D2, 3-O-25-hydroxyvitamin D2, 3-O-1-,25-dihydroxyvitamin D2, 3-O-vitamin D3, 3-O-1-hydroxyvitamin D3, 3-O-25-hydroxyvitamin D3, and/or 3-O-1-,25-dihydroxyvitamin D3), thyroxine and related analytes, estrogen and related analytes, and testosterone and related analytes.

The synthesis of the compound of Formula I having structure B is shown below in Scheme II.

Compound 1 is treated with sodium azide (NaN₃) in acetonitrile and heated to effect a Curtius rearrangement to provide the corresponding isocyante, which reacts with NH₂(CH₂)₃-((3)-O-Vitamin D) in a solvent (N,N-DMF or DMSO) to provide compound 3. Compound 3 can be purified by dialysis or other chromatographic techniques, for example, silica gel gravity column chromatography, reverse or normal phase HPLC or UPLC, or simple extraction/isolation techniques routinely used for isolation/purification for small to medium sized organic molecules. Other compounds having Formula I and structure B can be made by using the appropriate starting materials as known by those of skill in the art.

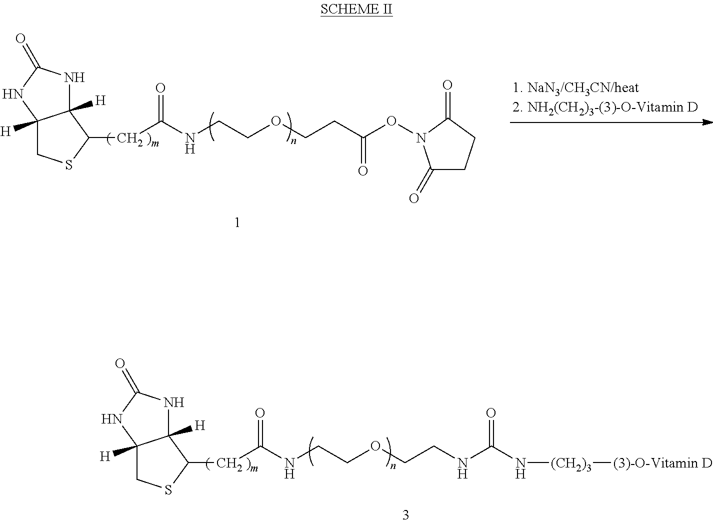

### Example III

### General Synthetic Procedures for the Synthesis of the Compounds of Formula I having Structure C

In one embodiment, the compound of Formula I has structure C:

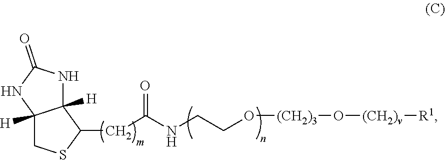

where m is an integer selected from 4, 5, and 6; n is an integer selected from 2 to 36; v is an integer selected from 1 to 6; and R¹ is 3-O-vitamin D (e.g., 3-O-vitamin D2, 3-O-1-hydroxyvitamin D2, 3-O-25-hydroxyvitamin D2, 3-O-1-,25-dihydroxyvitamin D2, 3-O-vitamin D3, 3-O-1-hydroxyvitamin D3, 3-O-25-hydroxyvitamin D3, and/or 3-O-1-,25-dihydroxyvitamin D3), thyroxine and related analytes, estrogen and related analytes, and testosterone and related analytes.

The synthesis of the compound of Formula I having structure C is shown below in Scheme III. Compound 4 can be prepared by base hydrolysis of compound 1 using LiOH in 1:1 ratio of H₂O and THF (H₂O:THF).

Compound 4 is converted to the corresponding methyl ester using methanol and a catalytic amount of sulfuric acid. The methyl ester is reduced to the corresponding alcohol using sodium borohydride in methanol. The alcohol is made into a leaving group by treatment with tosyl chloride in pyridine. Treatment of the tosylated intermediate with the sodium or potassium salt of HO—(CH₂)₃-(3)-O-Vitamin D in N,N-DMF provides compound 5. Compound 5 can be purified by dialysis or other chromatographic techniques, for example, silica gel gravity column chromatography, reverse or normal phase HPLC or UPLC, or simple extraction/isolation techniques routinely used for isolation/purification for small to medium sized organic molecules. Other compounds having Formula I and structure C can be made by using the appropriate starting materials as known by those of skill in the art.

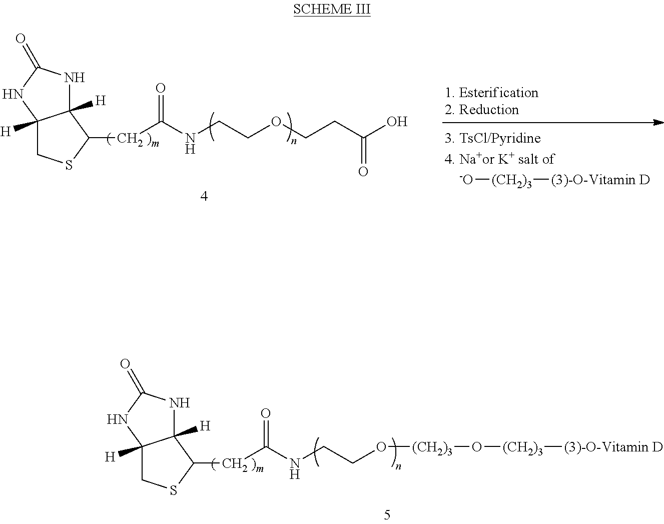

### Example IV

### General Synthetic Procedures for the Synthesis of the Compounds of Formula I having Structure D

In one embodiment, the compound of Formula I has structure D:

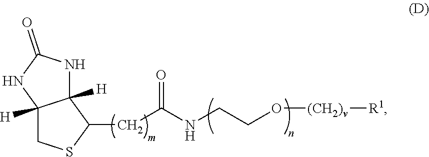

where m is an integer selected from 4, 5, and 6; n is an integer selected from 2 to 36; v is an integer selected from 1 to 6; and R¹ is 3-O-vitamin D (e.g., 3-O-vitamin D2, 3-O-1-hydroxyvitamin D2, 3-O-25-hydroxyvitamin D2, 3-O-1-,25-dihydroxyvitamin D2, 3-O-vitamin D3, 3-O-1-hydroxyvitamin D3, 3-O-25-hydroxyvitamin D3, and/or 3-O-1-,25-dihydroxyvitamin D3), thyroxine and related analytes, estrogen and related analytes, and testosterone and related analytes.

The synthesis of the compound of Formula I having structure D is shown below in Scheme IV. Compound 4 is selectively reduced and tosylated as described above. Treatment of the tosylated intermediate with 3-OH-Vitamin D in N,N-DMF provides compound 6. Compound 6 can be purified by dialysis or other chromatographic techniques, for example, silica gel gravity column chromatography, reverse or normal phase HPLC or UPLC, or simple extraction/isolation techniques routinely used for isolation/purification for small to medium sized organic molecules. Other compounds having Formula I and structure D can be made by using the appropriate starting materials as known by those of skill in the art.

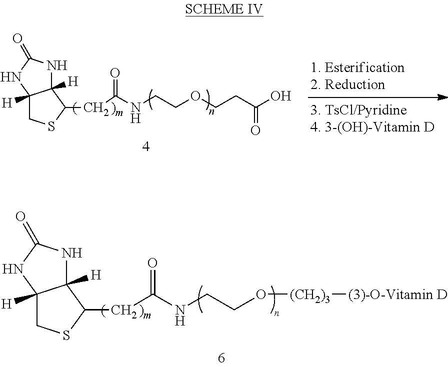

### Example V

### General Synthetic Procedures for the Synthesis of the Compounds of Formula I having Structure E

In one embodiment, the compound of Formula I has structure E:

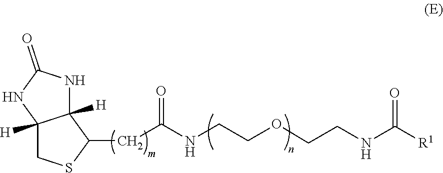

where m is an integer selected from 4, 5, and 6; n is an integer selected from 2 to 36; and R¹ is 3-O-vitamin D (e.g., 3-O-vitamin D2, 3-O-1-hydroxyvitamin D2, 3-O-25-hydroxyvitamin D2, 3-O-1-,25-dihydroxyvitamin D2, 3-O-vitamin D3, 3-O-1-hydroxyvitamin D3, 3-O-25-hydroxyvitamin D3, and/or 3-O-1-,25-dihydroxyvitamin D3), thyroxine and related analytes, estrogen and related analytes, and testosterone and related analytes.

The synthesis of the compound of Formula I having structure E is shown below in Scheme V. Compound 1 is treated with sodium azide (NaN₃) in acetonitrile and heated to effect a Curtius rearrangement to provide the corresponding isocyante, which reacts with 3-OH-Vitamin D in solvent (N,N-DMF or DMSO) to provide compound 7. Compound 7 can be purified by dialysis or other chromatographic techniques, for example, silica gel gravity column chromatography, reverse or normal phase HPLC or UPLC, or simple extraction/isolation techniques routinely used for isolation/purification for small to medium sized organic molecules. Other compounds having Formula I and structure E can be made by using the appropriate starting materials as known by those of skill in the art.

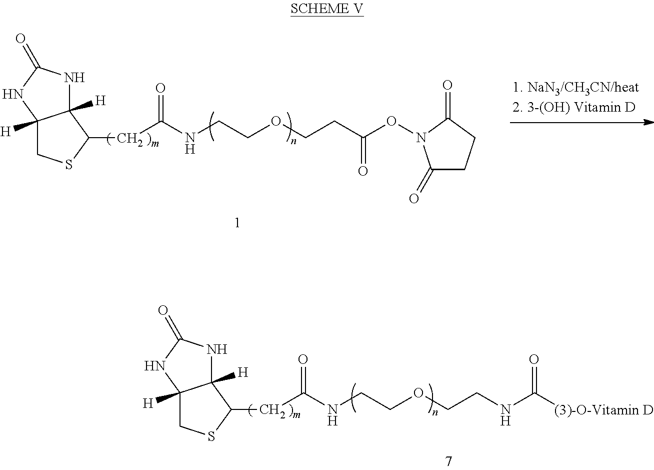

It is generally understood that the R¹ group in the above structures represents analytes which contain a hydroxyl, amino or thiol group. Such analytes can be selected from those described herein, however, they are not limited to only these.

In situations where the PEG spacer group is small for example, PEG2 through PEG10, the dialysis method for in situ purification of the biotin conjugate may not be viable. In these cases it is possible that the synthesized biotin analog can be isolated via chromatographic techniques like simple silica gel gravity column or reverse or normal phase HPLC or UPLC or simple extraction/isolation techniques routinely used for isolation/purification of small to medium sized organic molecules.

It is often necessary for the analyte precursors to be synthesized using routine organic chemistry techniques which may require the use and manipulation of function groups that could interfere with the desired synthetic outcome. In such case it is assumed that one of skill in the art could achieve the desired analyte precursor by selecting the appropriate protecting groups. One text book reference that illustrates and teaches the art of using and manipulating protecting groups in organic chemistry is that by authors Peter G. M. Wuts, Theodora W. Greene, titled Greene's Protective Groups in Organic Synthesis, John Wiley & Sons, Oct. 30, 2006.

### Example VI

### Method for Conjugating NH₂—(CH₂)₃-(3)-O-Vitamin D to Biotin-PEG 12-CO₂H

2.5 mg of NH₂—(CH₂)₃-(3)-O-Vitamin D was dissolved in 300 μl of methanol (8.3 mg/ml) and 25 mg of EZ-Link-NHS-PEG₁₂-biotin complex was dissolved in 1 ml of DMF (25 mg/ml). 50 μl of the NH₂—(CH₂)₃-(3)-O-Vitamin D solution was added to 1 ml of the EZ-Link NHS-PEG 12-biotin reagent solution. The reaction mixture was mixed by vortexing and was left in a rocker at low RPM for 30 to 50 minutes. The resulting mixture containing the product was dialyzed extensively against a 0.05 M PBS buffer solution (200 ml) using a dialysis membrane with MWCO=1K. The dialysis buffer was replaced four times every 2 to 3 hours and then left overnight at 4 C. After replacing the dialysis buffer once more in the morning and after 2 hours of further dialysis the dialyslate was isolated and its volume measured. Concentration=400 μg/2.5 ml vs. 160/ml (theoretical yield).

### Example VII

### General Method for an ELISA

The ELISA is intended for the quantitative determination of total 25-hydroxyvitamin D in human serum or plasma. A solid phase enzyme-linked immunoassay (ELISA) based on the principals of competitive binding is provided. Anti-vitamin D antibody coated wells are incubated with a vitamin D sample (control, serum or plasma sample) and a compound of Formula I at room temperature for 90 to 120 minutes. During the incubation, a fixed amount of the compound of Formula I competes with the endogenous vitamin D in the sample for a fixed number of binding sites on the anti-vitamin D antibody. Following a wash step, the bound compound of Formula I is detected with streptavidin-horse radish peroxidase (SA-HRP). The immunologically bound SA-HRP conjugated to the compound of Formula I progressively decreases as the concentration of vitamin D in the sample increases. Any unbound SA-HRP conjugated to the compound of Formula I is then removed and the wells are washed. Next, a solution of TMB (3,3′,5,5′-tetramethylbenzidine) reagent is added to the wells and incubated at room temperature for 30 minutes, resulting in the development of a blue color. The color development is stopped with the addition of stop solution (0.16N sulfuric acid), and the absorbance is measured spectrophotometrically at 450 nm. A standard curve is obtained by plotting the concentration of the standard versus the absorbance. The color intensity will be inversely proportional to the amount of 25-hydroxyvitamin D in the sample. The assay measures both the 25-hydroxyvitamin D2 and 25-hydroxyvitamin D3. The total assay procedure run time is 2.5 hours. The results are expressed in ng/mL. To convert to nmol/L, multiply results by 2.5 (e.g. 10 ng/ml=25 nmol/L). Each laboratory should use 25-hydroxyvitamin D controls to validate the performance of reagents and to establish the range of normal values that corresponds to the population of their region.

Serum, heparinized plasma or EDTA plasma samples can be used for the assay. For serum, collect whole blood by venipuncture and allow clotting. For plasma, mix the sample by gentle inversion prior to centrifugation. Centrifuge and separate serum or plasma as soon as possible after collection. Do not use hemolyzed samples. The specimens may be refrigerated at 2-8° C. for two weeks. For long term storage, they can be stored at −20° C. Avoid repeated freeze-thaw cycles. Allow the refrigerated or frozen-thawed samples to equilibrate to room temperature for 30 minutes before use; samples must be mixed before analysis.

Still other aspects, features, and technical effects will be readily apparent to those skilled in this art from the following detailed description, wherein embodiments are shown and described, simply by way of illustration of the best mode contemplated. The disclosure is capable of other and different embodiments, and its several details are capable of modifications in various obvious respects. Accordingly, the drawings and description are to be regarded as illustrative in nature, and not as restrictive.

The embodiments of the present disclosure can achieve several technical effects, including compounds, compositions and methods of treating bacterial infections using these compounds and compositions.

In the preceding description, the present disclosure is described with reference to specifically exemplary embodiments thereof. It will, however, be evident that various modifications and changes may be made thereto without departing from the broader spirit and scope of the present disclosure, as set forth in the claims. The specification and drawings are, accordingly, to be regarded as illustrative and not as restrictive. It is understood that the present disclosure is capable of using various other combinations and embodiments and is capable of any changes or modifications within the scope of the inventive concept as expressed herein.

1. A compound having Formula I: 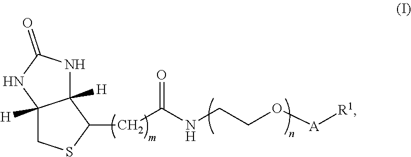
wherein:
A is —(CH₂)_{q}—, —(CH₂)_{q}O(CH₂)ₜ—, —(CH₂)_{q}NR²(CH₂)_{q}—, —(CH₂)_{q}S(O)ᵤ(CH₂)_{q}—, —(CH₂)ₜC(O)NR²(CH₂)ₜ—, —(CH₂)_{q}NR²C(O)(CH₂)ₜ—, —(CH₂)_{q}NR²C(O)O(CH₂)ₜ—, —(CH₂)_{q}NR²C(O)NR³(CH₂)ₜ—, or —(CH₂)_{q}NR²C(O)S(CH₂)_{q}—;
R¹ is vitamin D, thyroxine, estrogen or testosterone or a metabolite or derivative thereof;
R² and R³ are each independently hydrogen or (C₁-C₆)alkyl;
m is an integer number selected from 4, 5 and 6;
n is an integer number selected from 2 to 36;
q is an integer number selected from 1 to 12;
t is an integer number selected from 0 to 12; and
u is an integer number selected from 0, 1 and 2.

2. The compound of Formula I according to claim 1, wherein R¹ has Formula II: 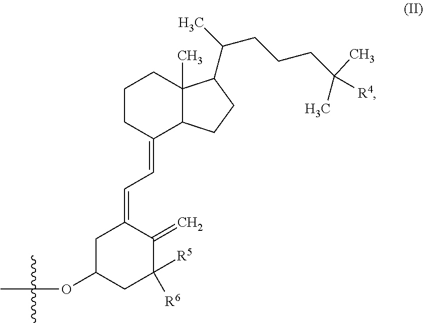
wherein:
R⁴ is hydrogen or hydroxyl; and
R⁵ and R⁶ are each independently hydrogen, hydroxyl, (C₁-C₆)alkyl, or (C₁-C₆)alkoxy.

3. The compound of Formula I according to claim 1, wherein R¹ is 25-hydroxyvitamin D₂ or 25-hydroxyvitamin D₃.

4. The compound of Formula I according to claim 1, wherein R¹ has Formula III: 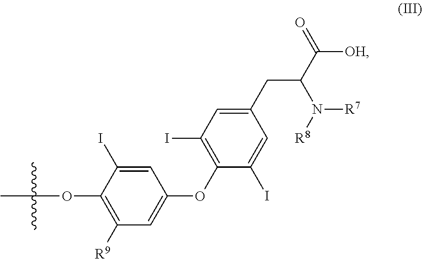
wherein:
R⁷ and R⁸ are each independently hydrogen or (C₁-C₆)alkyl; and
R⁹ is hydrogen or iodine, or
R^{l} has Formula IV: 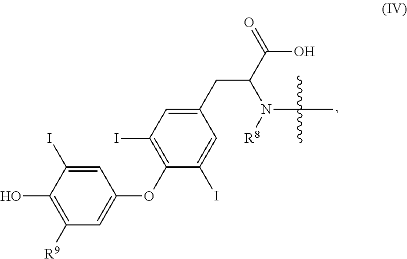
wherein:
R⁷ is hydrogen or (C₁-C₆)alkyl; and
R⁹ is hydrogen or iodine.

5. The compound of Formula I according to claim 1, wherein R¹ is triidothyronine (T₃) or thyroxine (T₄).

6. The compound of Formula I according to claim 1, wherein R¹ has Formula V, VI, VII, VIII, IX, X, or XI:

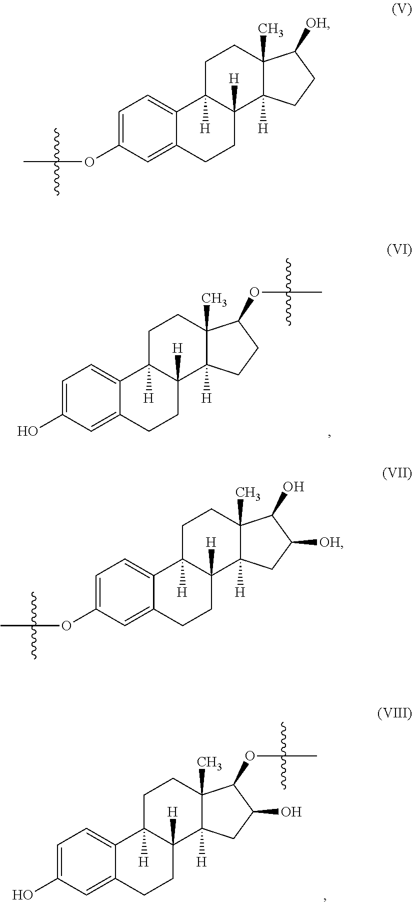

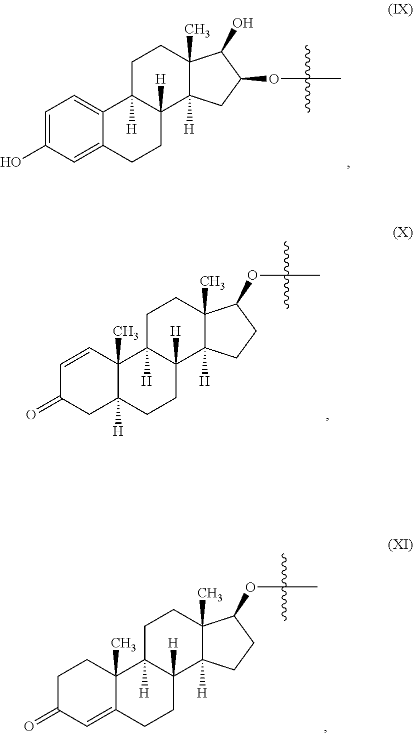

7. The compound of Formula I according to claim 1, wherein R¹ is estrone (E₁), estradiol (E₂), estriol (E₃), 1-testosterone (1-T), or testosterone.

8. The compound of Formula I according to claim 1, wherein A is —(CH₂)₃—, —(CH₂)₃O(CH₂)₃₋₆—, —(CH₂)₃NR²(CH₂)₃₋₆—, —(CH₂)₃S(CH₂)₃₋₆—, —(CH₂)₂C(O)NR²(CH²)₃₋₆—, —(CH₂)_{q}NR²C(O)(CH₂)₃₋₆—, —(CH₂)_{q}NR²C(O)O(CH₂)₃₋₆—, —(CH₂)_{q}NR²C(O)S(CH₂)₃₋₆—, —(CH₂)_{q}NR²C(O)NR²(CH₂)₃₋₆—, or —(CH₂)₂NR²C(O)—.

9. A solid phase assay method for detecting vitamin D, thyroxine, estrogen or testosterone or a metabolite or derivative thereof in a sample, comprising:
a) providing a solid phase support having immobilized thereon a protein or antibody that binds to the vitamin D, thyroxine, estrogen or testosterone or a metabolite or derivative thereof in the sample, and a compound having Formula I: 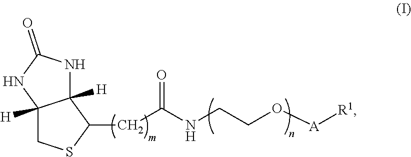
wherein:
A is —(CH₂)_{q}—, —(CH₂)_{q}O(CH₂)ₜ—, —(CH₂)_{q}NR²(CH₂)_{q}—, —(CH₂)_{q}S(O)ᵤ(CH₂)_{q}—, —(CH₂)ₜC(O)NR²(CH₂)ₜ—, —(CH₂)_{q}NR²C(O)(CH₂)ₜ—, —(CH₂)_{q}NR²C(O)O(CH₂)ₜ—, —(CH₂)_{q}NR²C(O)NR³(CH₂)ₜ—, or —(CH₂)_{q}NR²C(O)S(CH₂)_{q}—;
R¹ is vitamin D, thyroxine, estrogen or testosterone or a metabolite or derivative thereof;
R² and R³ are each independently hydrogen or (C₁-C₆)alkyl;
m is an integer number selected from 4, 5 and 6;
n is an integer number selected from 2 to 36;
q is an integer number selected from 1 to 12;
t is an integer number selected from 0 to 12; and
u is an integer number selected from 0, 1 and 2;
b) contacting the solid phase support with the sample of the vitamin D, thyroxine, estrogen or testosterone or a metabolite or derivative thereof, and the compound of Formula I, for a time sufficient to allow binding to the protein or antibody attached to the solid phase support;
c) contacting the solution and solid phase support obtained in step (b) with streptavidin-horse radish peroxidase (SA-HRP), for a time sufficient to effect conjugation of the SA-HRP to the compound of Formula I, which may be unbound or bound to the protein or antibody attached to the solid phase support;
d) removing the solution obtained in step (c) containing unbound SA-HRP conjugated to the compound of Formula I; and
e) detecting the presence of bound SA-HRP conjugated to the compound of Formula I,
wherein the amount of the bound SA-HRP conjugated to the compound of Formula I in step e) is inversely proportional to the amount of the vitamin D, thyroxine, estrogen or testosterone or a metabolite or derivative thereof in the test sample.

10. The solid phase assay method according to claim 9, wherein the assay is for vitamin D and wherein the vitamin D is 25-hydroxyvitamin D2 or 25-hydroxyvitamin D3.

11. The solid phase assay method according to claim 9, comprising detecting the presence of bound SA-HRP conjugated to the compound of Formula I by absorbance measured spectrophotometrically at 450 nm.

12. The solid phase assay method of claim 11, comprising adding a solution of TMB (3,3′,5,5′-tetramethylbenzidine) reagent to the bound SA-HRP conjugated to the compound of Formula I and incubating at room temperature to develop a blue color; stopping the blue color development by the addition of a stop solution (0.16N sulfuric acid); and measuring absorbance spectrophotometrically at 450 nm against a standard curve.

13. A kit for determining the concentration of vitamin D, thyroxine, estrogen or testosterone or a metabolite or derivative thereof in a sample of human serum by an immune-based competitive protein binding assay, comprising:
a) a standardized quantity of a compound of Formula I or a standardized solution of a compound of Formula I: 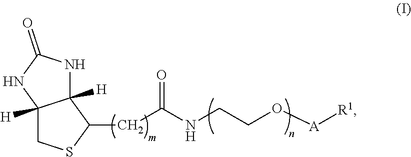
wherein:
A is —(CH₂)_{q}—, —(CH₂)_{q}O(CH₂)ₜ—, —(CH₂)_{q}NR²(CH₂)_{q}—, —(CH₂)_{q}S(O)ᵤ(CH₂)_{q}—, —(CH₂)ₜC(O)NR²(CH₂)ₜ—, —(CH₂)_{q}NR²C(O)(CH₂)ₜ—, —(CH₂)_{q}NR²C(O)O(CH₂)ₜ—, —(CH₂)_{q}NR²C(O)NR³(CH₂)ₜ—, or —(CH₂)_{q}NR²C(O)S(CH₂)_{q}—;
R¹ is vitamin D, thyroxine, estrogen or testosterone or a metabolite or derivative thereof;
R² and R³ are each independently hydrogen or (C₁-C₆)alkyl;
m is an integer number selected from 4, 5 and 6;
n is an integer number selected from 2 to 36;
q is an integer number selected from 1 to 12;
t is an integer number selected from 0 to 12; and
u is an integer number selected from 0, 1 and 2; and
b) a standardized quantity of a protein or antibody that specifically binds to vitamin D, thyroxine, estrogen or testosterone or a metabolite or derivative thereof, and to the compound having Formula I, or a pharmaceutically acceptable salt or hydrate thereof.

14. The kit of claim 13, comprising a solid phase support having immobilized thereon the standardized quantity of a protein or antibody that specifically binds to vitamin D, thyroxine, estrogen or testosterone or a metabolite or derivative thereof, and to the compound having Formula I.

15. The kit of claim 14, wherein the solid phase support is selected from the group consisting of a microtitration plate, a microparticle, a polymeric material, and cellulose.

16. The kit of claim 13, further comprising streptavidin-horse radish peroxidase (SA-HRP).

17. The kit of claim 13, wherein the competitive protein binding assay is an enzyme immunoassay, an enzyme-linked immunosorbent assay, a radioimmunoassay, an immunoradiometric assay, a luminescence assay, a fluorescence immunoassay or an immunofluorometric assay.

18. The kit of claim 13, wherein the competitive protein binding assay is an enzyme-linked immunosorbent assay.
